(19) United States
(12) Patent Application Publication
(10) Pub. No.: US 2025/0134372 A1
(43) Pub. Date: May 1, 2025

(54) One Gram Penny-Sized Wireless EEG Recording Patch for Long-Term Mental Health Monitoring

(71) Applicants: THE BOARD OF TRUSTEES OF THE LELAND STANFORD JUNIOR UNIVERSITY, Stanford, CA (US); CZ BIOHUB SF, LLC., San Francisco, CA (US)

(72) Inventors: Ada Shuk Yan Poon, Stanford, CA (US); Cheng Chen, Cupertino, CA (US)

(21) Appl. No.: 18/835,705

(22) PCT Filed: Feb. 3, 2023
(86) PCT No.: PCT/US2023/012268

§ 371 Date: Aug. 2, 2024

Related U.S. Application Data
(60) Provisional application No. 63/307,906, filed on Feb. 8, 2022.

(51) Int. Cl. A61B 5/00; A61B 5/257; A61B 5/372; A61B 5/384

(52) U.S. Cl. CPC A61B 5/0006; A61B 5/257; A61B 5/372; A61B 5/384; A61B 2560/0214

(57) ABSTRACT

A wireless electroencephalogram (EEG) recording system is provided, comprising: one or more two-channel electrodes; an analog-to-digital converter (ADC) configured to capture EEG data detected by the one or more electrodes; a digital controller configured to encode the captured EEG data into a single-bit series, and generate a packet using the single-bit series; and a radio frequency (RF) transmitter configured to transmit the packet to an external receiver. In some examples, the wireless EEG recording system further includes a battery powering the one or more ADCs, the digital controller, and the RF transmitter. For instance, in some examples, the wireless EEG recording system further includes an adhesive patch configured to be attached to the head of a patient, wherein the one or more two-channel electrodes, a chip including the ADC, the digital controller and the RF transmitter, and the battery are each attached to the adhesive patch.

### FIELD OF THE DISCLOSURE

The present disclosure generally relates to electroencephalogram (EEG) recording and, more particularly, to a lightweight, one gram wireless EEG recording system.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

As a non-invasive brain wave monitoring method, electroencephalogram (EEG) recording has been widely used in the fields of epilepsy detection, sleep disorder diagnosis, psychiatric conditions study, etc., by recording and analyzing surface signals of more than a dozen sites on human head. Conventional EEG instruments are heavy and use bulky wires to deploy multiple electrodes, causing discomfort to the subject. This limits the group of applicable subjects, as well as their longitudinal studies that require long-term continuous monitoring of brain activities.

### SUMMARY

In one aspect, a wireless electroencephalogram (EEG) recording system is provided herein, comprising: one or more two-channel electrodes; an analog-to-digital converter (ADC) configured to capture EEG data detected by the one or more electrodes; a digital controller configured to encode the captured EEG data into a single-bit series, and generate a packet using the single-bit series; and a radio frequency (RF) transmitter configured to transmit the packet to an external receiver. The wireless EEG recording system provided herein may be as light as 1 gram in weight.

In some examples, the wireless EEG recording system further includes a battery powering the one or more ADCs, the digital controller, and the RF transmitter. For instance, in some examples, the wireless EEG recording system further includes an adhesive patch configured to be attached to the head of a patient, wherein the one or more two-channel electrodes, a chip including the ADC, the digital controller and the RF transmitter, and the battery are each attached to the adhesive patch. In some examples, the battery operates using 1.2-1.8 V. Moreover, in some examples, the battery provides power at a rate greater than or equal to 100 μW. Additionally, in some examples, the battery has a total load capacity less than or equal to 120 mWh.

Furthermore, in some examples, each recording channel, of the two-channel electrodes, is a 12-bit, 33-kHz sigma-delta ADC channel.

Additionally, in some examples, each two-channel electrode is positioned less than 2 cm from each other.

Moreover, in some examples the RF transmitter transmits the packet to the external receiver using an industrial, scientific, and medical (ISM) band. For instance, in some examples, the transmitter operates in a range of 902 MHz to 928 MHz. Additionally, in some examples, the transmitter supports twelve wireless EEG recording devices within the ISM band.

Furthermore, in some examples, the RF transmitter transmits the packet to the external receiver using less than or equal to 200 Kbps.

Additionally, in some examples, the wireless EEG recording device is located a distance less than or equal to 10 m from the external receiver.

### BRIEF DESCRIPTION OF THE DRAWINGS

FIG. 1 illustrates a system diagram, and photo of, a two-channel frequency division multiple access (FDMA) electroencephalogram (EEG) patch, using a size-10 hearing aid battery, in accordance with some embodiments provided herein.

FIG. 2 illustrates an FDMA TX with a low power ring-oscillator-based integer-N frequency synthesizer, in accordance with some embodiments provided herein. As shown in FIG. 2, discrete tuning of VCO supply voltage compensates process and temperature variations.

FIGS. 3A-3C illustrate a FDMA TX performance characterization, including: at FIG. 3A, a spectrum of single channel, at FIG. 3B, a spectrum when 4 devices transmitting concurrently, and, at FIG. 3C, an external receiver (RX) down-conversion of data in adjacent channels, in accordance with some embodiments provided herein.

FIG. 4 illustrates the architecture of a sigma-delta (Σ-Δ) ADC and data packet generator, as well as a graph illustrating ADC signal to noise and distortion ratio (SNDR) vs input voltage, in accordance with some embodiments provided herein. As shown in FIG. 4, the ADC has a peak signal to noise and distortion ratio SNDR of 58.1 dB, translating to 9.4 effective number of bits (ENOB).

FIGS. 5A and 5B illustrate a comparison of EEG recordings, between an FDMA EEG patch, in accordance with some embodiments provided herein, and a clinical instrument. FIG. 5A illustrates a comparison of EEG recordings between the FDMA EEG patch and the clinical instrument for an eye-closed (spectral) test, while FIG. 5B illustrates a comparison of EEG recordings between the FDMA EEG patch and the clinical instrument for an oddball (temporal) test. As shown at FIGS. 5A and 5B, similar performances have been observed for both the eye-closed test and the oddball test.

FIG. 6 is a performance comparison table illustrating various performance parameters for the FDMA EEG patch, in accordance with some embodiments provided herein, several other EEG recording devices, including a power breakdown of each block.

FIG. 7 illustrates a die micrograph for the FDMA EEG patch, in accordance with some embodiments provided herein. The chip area for the die micrograph shown in FIG. 7 is 1.65×1.53 mm².

### DETAILED DESCRIPTION

As discussed above, electroencephalogram (EEG) recording has been widely used in the fields of epilepsy detection, sleep disorder diagnosis, psychiatric conditions study, etc., by recording and analyzing surface signals of more than a dozen sites on human head. However, conventional EEG instruments use bulky wires to deploy multiple electrodes, causing discomfort to the subject. This limits the group of applicable subjects, as well as their longitudinal studies that require long-term continuous monitoring of brain activities. Therefore, a small-profile, wearable EEG recorder capable of capturing clinically valid signals without user discomfort is in demand.

Conventional commercially available EEG systems, such as commercially available sleep monitoring wearable devices, have only been capable of recording data on limited locations such as the patient's forehead and ear, and cannot record any data on the large area of the upper head including the parietal region. The electrodes are also very painful, and the device is bulky to be fixed firmly on the head. Thus, the acquired data was not clinically useful. Moreover, a single-channel device cannot detect a signal that is perpendicular to the line of measurement.

Most clinically graded EEG devices come with heavily wired recording systems ranging from 16 to 128+ channels. Although these devices provide the highest data quality, they can be only used in controlled lab environment. Also due to the bulkiness of the system, usually the subjects cannot wear it for more than an hour.

Recently, an in-ear wireless EEG recorder has been demonstrated with Bluetooth® low energy (BLE) for data telemetry. However, this system's power efficiency is limited by the general-purpose radio module, and it cannot reach established recording sites without long wires.

A concurrent recording and transmitting active EEG electrode utilizing body channel communication (BCC) has also been proposed. However, for the concurrent recording and transmitting active EEG electrode utilizing BCC, each device is supplied by a bulky quarter-size coin battery, and a specialized receiver (RX) needs to be attached to the head.

A radio frequency (RF)-powered FDD radio for neural microimplants has also been proposed, in which case the low-power transmitter (TX) potentially enables the use of pea-size hearing-aid battery for long-term recording, but the limited phase noise performance prevents multi-access protocols needed for concurrent transmission by multiple wireless recorders, confining the measurement to single location, which is unfavored in analyzing brain activities.

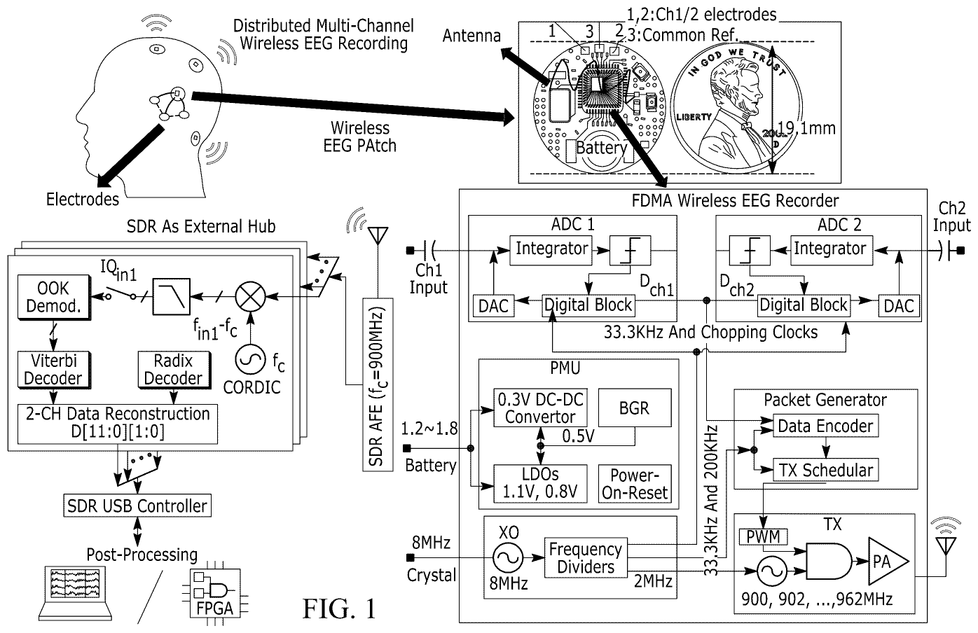

In contrast to these conventional approaches, the present disclosure provides a lightweight (about 1 gram) frequency division multiple access (FDMA) wireless EEG recording device in 902-928 MHz industrial, scientific and medical (ISM) band with concurrent data telemetry for up to 12 devices with a month-long battery life using only a size-10 hearing aid battery (ϕ=5.8 mm, h=3.6 mm), as shown at FIG. 1. Advantageously, the wireless EEG recording device is light enough to use wet adhesives and electrodes which are very comfortable to skin, and the recording patch can be deployed anywhere on the head. Each EEG patch measures two bipolar, localized EEG signals where two electrodes with a common electrode are placed orthogonally within 2 cm of distance. As low-profile wearable recorder necessitates locally deployed electrodes, the measured signal is essentially a surface gradient of a scalp potential (i.e., electric field) which is two-dimensional in nature. That is, adopting two spatially orthogonal channels per patch allows the wireless EEG patch provided herein to capture the scalp signals that are inherently two-dimensional. Accordingly, the two spatially orthogonal channels successfully capture these local electric fields, whereas single channel methods that have been attempted by others fail to capture signal perpendicular to its measurement direction. The recorder consists of two sigma-delta (Σ-Δ) ADCs, where a customized micro-controller combines the recorder data and generates data packets for the TX, which has a selectable carrier frequency from 904, 906, etc., up to 926 MHz. The power amplifier (PA) output power can be programmed between-32 dBm and-18 dBm, and the PA is supplied by a DC-DC converter for maximum efficiency. A total system power of 90 μW is achieved with 1.5 V supply when PA is in its lowest power mode, translating to >1000 h battery life with size-10 hearing aid-battery. Portable external receiver setup consists of an antenna, a software-defined radio (SDR), and a computer or a field programmable gate array (FPGA).

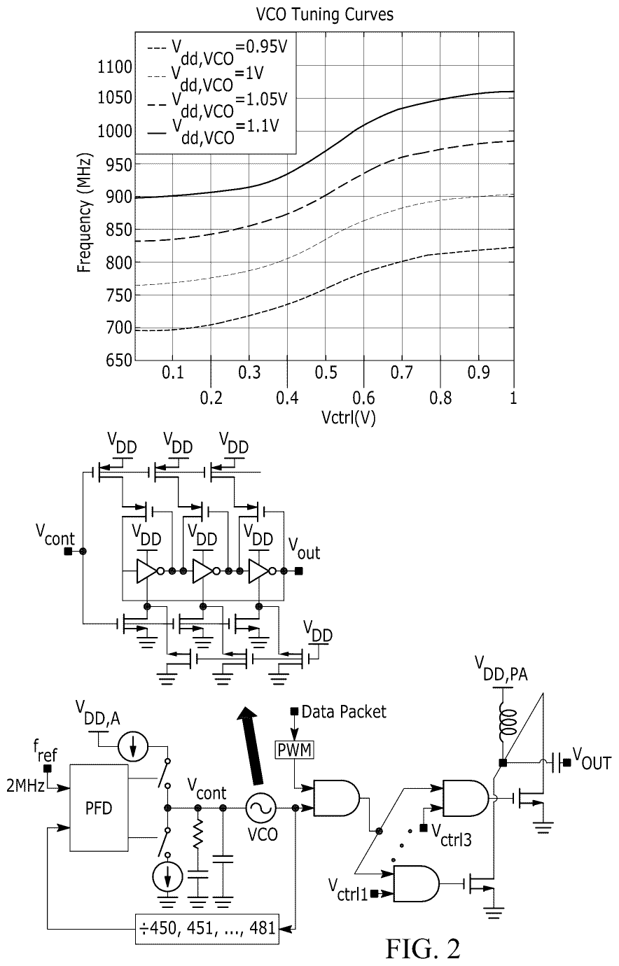

A type-II phase locked loop (PLL) based integer-N frequency synthesizer is the core of the FDMA TX, as shown at FIG. 2. The 2 MHz reference clock is divided from an 8 MHz on-chip crystal oscillator (XO). On-off keying (OOK) modulation is chosen as it directly trades off the minimum requirement on frequency synthesizer phase noise for lower voltage-controlled oscillator (VCO) power consumption. As a result, the requirement on frequency synthesizer phase noise is only determined by inter-channel interference (151). Multiple other techniques have also been deployed together to minimize the TX power consumption. Ring-oscillator is chosen over LC oscillator in VCO design as it can achieve a lower power consumption while still meeting phase noise requirement. Source degenerations for n-channel metal-oxide semiconductor (NMOS) transistor and tunable positive feedback for p-channel metal oxide semiconductor (PMOS) transistor are used for symmetric frequency tuning across control voltage range. VCO supply is generated by a dedicated programmable LDO so that it can be trimmed across process corners, as shown at FIG. 2. True-single-phase clock (TSPC) logic, together with a pulse-swallow integer-N divider further reduces the TX power consumption. Programmable TX output is achieved by tuning either its supply or the size of its driver transistor, thus enabling minimum PA power consumption for the application range. The low PA supply voltage ranging from 0.3-0.6 V is generated from a programmable-output buck converter, further improving the system power efficiency compared with using an LDO.

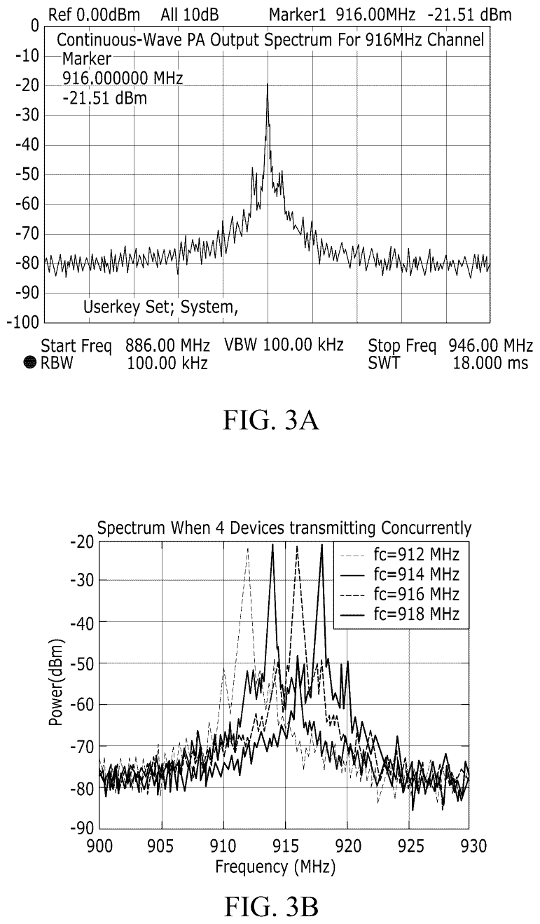

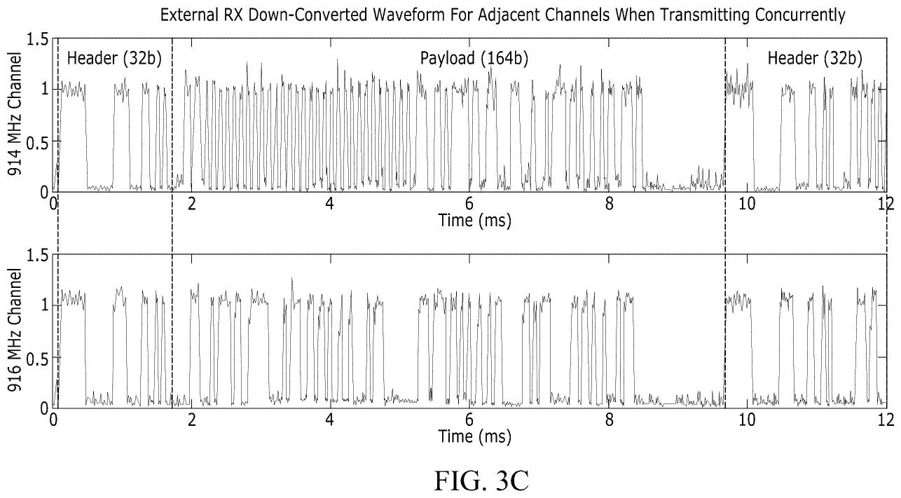

FIGS. 3A-3B illustrate the TX measurement results. A phase noise of −85.4 dBc/Hz at 1 MHz offset is obtained with 13.5 μA current on 0.95 V power supply, translating to a figure of merit (FOM) of 164 (mW·dBc/Hz)⁻¹. The reconstructed channel spectrum plot shows minimum inter-channel interference. To demonstrate the FMDA capability, transient waveforms of two EEG patches concurrently transmitting at 914 MHz and 916 MHz are shown. Further analysis shows that the bit error rate (BER) is less than 10⁻⁶. The buck converter that supplies the PA achieves an output voltage ripple of 3 mV and the quiescent current consumption is 800 nA, resulting in an 80% overall efficiency with 10 μW load.

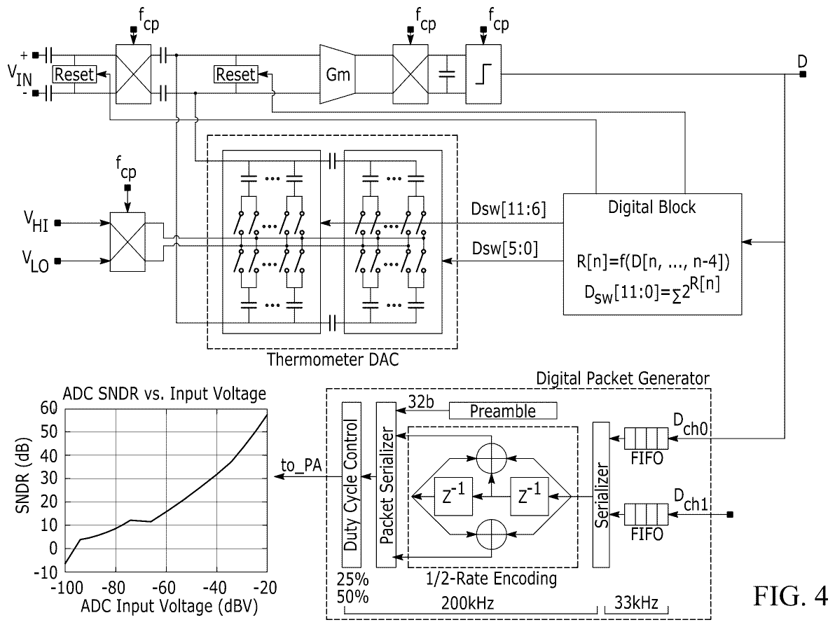

The recorder uses two 12-bit, 33-kHz Σ-Δ ADCs as shown at FIG. 4, followed by a microcontroller where each channel output is grouped into 32 bits, convolutional encoded and then packetized with a preamble header. Then the output packet's duty cycle is selected from 25% and 50% to reduce power consumption of the power amplifier. The ADC takes differential inputs, AC-coupled using 10-nF on-board capacitors. Three choppers, located at recorder input, output of the operational transconductance amplifier (OTA) and digital-to-analog (DAC) reference voltage input, suppress the flicker noise as the signal of interest is down to 0.5 Hz. The digital block employs a radix-based range prediction algorithm to convert output into a single bit without compromising the data rate. It also generates a reset signal to enable fast settling when 7 consecutive 1 s arrive from the comparator, indicating an exponential growth of coded value. In presence of large instantaneous artifact, the digital block turns on the reset switches and achieve fast recovery. A serial DAC with 6b+6b architecture is designed to reduce power consumption. Together with the elimination of a pre-amplifier, the 12-bit Σ-Δ ADC can achieve a low power consumption of 2 μW per channel. The recorder dynamic range is trimmed with reference voltage settings. As shown in the signal to noise and distortion ratio (SNDR) vs input amplitude measurement, a peak effective number of bits (ENOB) of 9.4 bits is achieved with 1 mV input

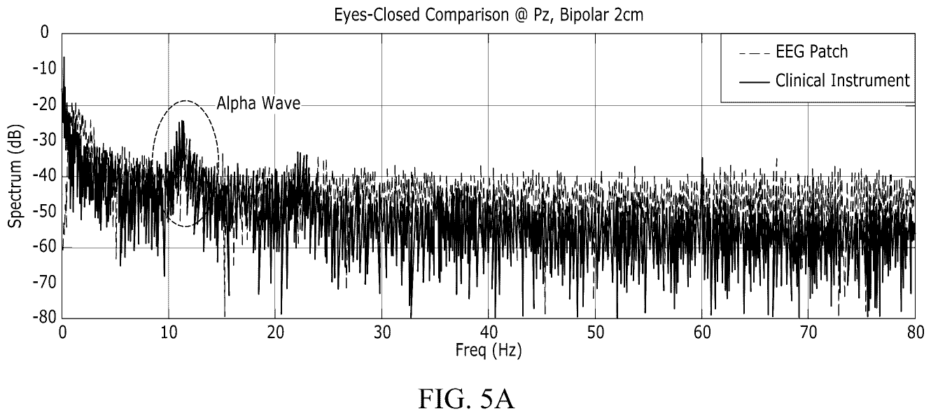

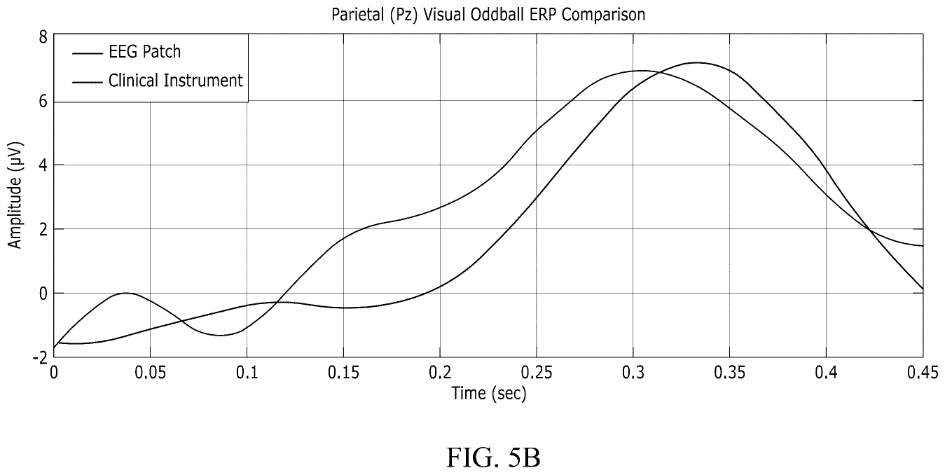

To validate the functionality of the wireless EEG patch, the wireless EEG patch was compared to ActiCHamp™ from Brain Vision, a clinically-graded device. FIGS. 5A and 5B illustrate spectral and temporal measurements of commonly-used EEG signals, respectively, measured locally at the parietal region (Pz) within 2 cm range. FIG. 5A shows a clearly visible peak at the alpha band (7-12 Hz) during an eyes-closed state. FIG. 5B illustrates a transient response to a visual oddball event-related potential (ERP) test at the Pz, compared with the clinically-graded device. This demonstrates that the EEG patch could successfully detect P300 peak, which is an important indicator for a cognitive response to stimulus after about 300 ms.

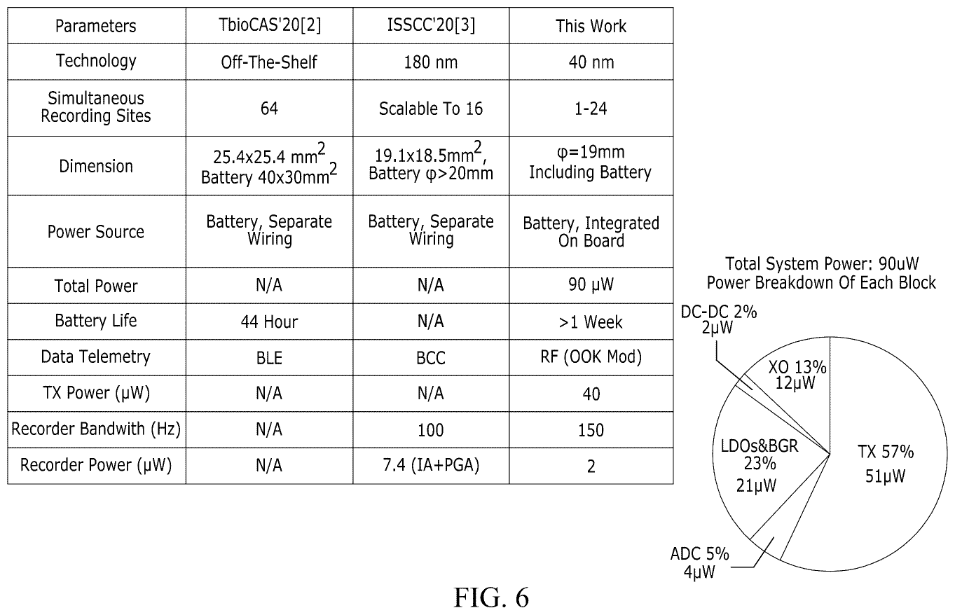

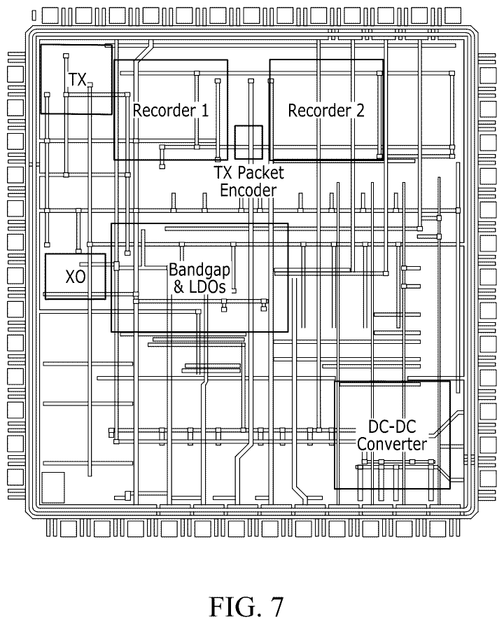

The performance summary is shown at FIG. 6 and the chip consumes 90 μW power, which, for instance, can last for up to a month using a small hearing-aid battery. The proposed FDMA wireless 2-channel EEG patch is fabricated in 40 nm 1P9M complementary metal-oxide-semiconductor (CMOS) process. The die micrograph is shown at FIG. 7. Combining the advantage of both low-power Σ-Δ ADC and OOK TX, a month-long multi-device wireless EEG recording may be achieved with only the size of a US penny including the battery.

Bluetooth® and WiFi are two standardized wireless communication protocols utilizing the 2.4 GHz ISM band. Bluetooth® is further divided into two subcategories: Bluetooth® classic and BLE. In general, the power consumption of Bluetooth® classic and WiFi is larger than 10 mW and 1 W, respectively, unsuitable for long-term continuous monitoring of low-frequency biophysical signals with battery power. On the other hand, BLE, initially designed to focus on the Internet of Things (IoT) applications with lower-speed and/or intermittent communication, consumes approximately or less than 10 mW power during data transfer. As a result, BLE transceivers (TRX) are the most common radio modules found in today's wireless bio-signal recorders using off-the-shelf components.

However, for a sub-10 g (weight) miniaturized wireless EEG patch, the largest battery capacity available is size-10 hearing-aid battery, rated as 1.45 V (actual voltage in measurement is 1.25 V) and 95 mAh, equivalently around 120 mWh. Total power consumption of less than 160 W is needed to support a continuously running device for more than a month on the battery. This is at least an order of magnitude smaller than BLE power consumption.

As disclosed herein, a 90 uW custom-designed application-specific integrated circuit (ASIC) is provided for wireless EEG recording with a proprietary communication protocol. There are several differences between this design and an off-the-shelf BLE radio module that enables this order-of-magnitude power reduction.

One difference between this design and an off-the-shelf BLE radio module is that the protocol provided herein uses 915 MHz ISM band (902-928 MHz), whereas BLE uses 2.4 GHz ISM band (2.4-2.5 GHZ). The 2.4-2.5 GHZ ISM band supports more channels due to the larger total bandwidth: 40 channels for BLE vs. 12 channels for the protocol provided herein. However, the 2.4 GHz ISM band has higher path loss (proportional to f\hat{ }2), which requires higher power amplifier (PA) power to compensate. 12 wireless channels, with each device having 2 or 4 recorders, result in total 24 or 48 EEG recording channels simultaneously, which are enough for various standard EEG applications.

Another difference between this design and an off-the-shelf BLE radio module is that the maximum data rate BLE supports in each channel is 2 Mbps, whereas it is 200 Kbps for the protocol provided herein. Higher data rate in BLE, together with the modulation method (GFSK) requires better oscillator phase noise performance, which directly trades off with power consumption. With 1/10th data rate and OOK modulation, the radio provided herein can be designed with considerably less oscillator/frequency synthesizer power (>300 μW for BLE vs. ˜30 μW). 200 Kbps is enough for most bio-signal acquisition application, including EEG recording, as the signal of interest is generally below 1 KHz.

Moreover, another difference between this design and an off-the-shelf BLE radio module is that BLE radio needs to support up to 30 m distance, requiring a maximum PA output power of 0 dBm (1 mW), whereas the radio provided herein has −30-−20 dBm PA output power. As provided herein, the PA output power, although 2 to 3 orders of magnitude smaller than maximum value of a typical BLE radio, is enough for 5˜10 m distance. Reducing the PA output power (backing-off) of a BLE radio will result in great reduction of its efficiency. Therefore, a BLE radio operating with −30 dBm-−20 dBm PA output power consumes 1 to 2 orders of magnitude power than the PA provided herein.

Furthermore, another difference between this design and an off-the-shelf BLE radio module is that BLE radios, to support various devices and to satisfy general privacy requirement, has complex “pairing” /connection protocols and encryption algorithms. This results in higher latency in data transmission and higher power consumption in packet generation. On the contrary, the protocol provided herein is specifically designed between the ASIC and external receiver (RX) provided herein, resulting in less complexity or redundancy.

Advantageously, the wireless EEG recording device provided herein may be directly commercialized into a wearable EEG recorder product aimed at any general public willing to monitor the brain's mental health on a long-term, daily basis. Beneficially, clinical researchers can also use the wireless EEG recording device provided herein to conduct longitudinal studies on a subject's EEG conditions outside of the clinic.

### Additional Considerations

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter of the present disclosure.

Additionally, certain embodiments are described herein as including logic or a number of components, modules, or mechanisms. Modules may constitute either software modules (e.g., code stored on a machine-readable medium) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

A hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module in dedicated and permanently configured circuitry or in temporarily configured circuitry (e.g., configured by software) may be driven by cost and time considerations.

Accordingly, the term hardware should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware and software modules can provide information to, and receive information from, other hardware and/or software modules. Accordingly, the described hardware modules may be regarded as being communicatively coupled. Where multiple of such hardware or software modules exist contemporaneously, communications may be achieved through signal transmission (e.g., over appropriate circuits and buses) that connect the hardware or software modules. In embodiments in which multiple hardware modules or software are configured or instantiated at different times, communications between such hardware or software modules may be achieved, for example, through the storage and retrieval of information in memory structures to which the multiple hardware or software modules have access. For example, one hardware or software module may perform an operation and store the output of that operation in a memory device to which it is communicatively coupled. A further hardware or software module may then, at a later time, access the memory device to retrieve and process the stored output. Hardware and software modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as an SaaS. For example, as indicated above, at least some of the operations may be performed by a group of computers (as examples of machines including processors), these operations being accessible via a network (e.g., the Internet) and via one or more appropriate interfaces (e.g., APIs).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” or a “routine” is a self-consistent sequence of operations or similar processing leading to a desired result. In this context, algorithms, routines and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Upon reading this disclosure, those of skill in the art will appreciate still additional alternative structural and functional designs through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

### Aspects

1. A wireless electroencephalogram (EEG) recording system, comprising: one or more two-channel electrodes; an analog-to-digital converter (ADC) configured to capture EEG data detected by the one or more electrodes; a digital controller configured to encode the captured EEG data into a single-bit series, and generate a packet using the single-bit series; and a radio frequency (RF) transmitter configured to transmit the packet to an external receiver.
2. The wireless EEG recording system of aspect 1, further comprising a battery powering the one or more ADCs, the digital controller, and the RF transmitter.
3. The wireless EEG recording system of aspect 2, further comprising an adhesive patch configured to be attached to the head of a patient, wherein the one or more two-channel electrodes, a chip including the ADC, the digital controller and the RF transmitter, and the battery are each attached to the adhesive patch.
4. The wireless EEG recording device of aspect 2, wherein the battery operates using 1.2-1.8 V.
5. The wireless EEG recording device of aspect 2, wherein the battery provides power at a rate greater than or equal to 100 μW.
6. The wireless EEG recording device of aspect 2, wherein the battery has a total load capacity less than or equal to 120 mWh.
7. The wireless EEG recording device of aspect 1, wherein each recording channel, of the two-channel electrodes, is a 12-bit, 33-kHz sigma-delta ADC channel.
8. The wireless EEG recording device of aspect 1, wherein each two-channel electrode is positioned less than 2 cm from each other.
9. The wireless EEG recording device of aspect 1, wherein the RF transmitter transmits the packet to the external receiver using an industrial, scientific, and medical (ISM) band.
10. The wireless EEG recording device of aspect 9, wherein the transmitter operates in a range of 902 MHz to 928 MHz.
11. The wireless EEG recording device of aspect 9, wherein the transmitter supports twelve wireless EEG recording devices within the ISM band.
12. The wireless EEG recording device of aspect 1, wherein the RF transmitter transmits the packet to the external receiver using less than or equal to 200 Kbps.
13. The wireless EEG recording device of aspect 1, wherein the wireless EEG recording device is located a distance less than or equal to 10 m from the external receiver.

1. A wireless electroencephalogram (EEG) recording system, comprising:
one or more two-channel electrodes;
an analog-to-digital converter (ADC) configured to capture EEG data detected by the one or more electrodes;
a digital controller configured to encode the captured EEG data into a single-bit series, and generate a packet using the single-bit series; and
a radio frequency (RF) transmitter configured to transmit the packet to an external receiver.

2. The wireless EEG recording system of claim 1, further comprising a battery powering the one or more ADCs, the digital controller, and the RF transmitter.

3. The wireless EEG recording system of claim 2, further comprising an adhesive patch configured to be attached to the head of a patient, wherein the one or more two-channel electrodes, a chip including the ADC, the digital controller and the RF transmitter, and the battery are each attached to the adhesive patch.

4. The wireless EEG recording device of claim 2, wherein the battery operates using 1.2-1.8 V.

5. The wireless EEG recording device of claim 2, wherein the battery provides power at a rate greater than or equal to 100 μW.

6. The wireless EEG recording device of claim 2, wherein the battery has a total load capacity less than or equal to 120 mWh.

7. The wireless EEG recording device of claim 1, wherein each recording channel, of the two-channel electrodes, is a 12-bit, 33-kHz sigma-delta ADC channel.

8. The wireless EEG recording device of claim 1, wherein each two-channel electrode is positioned less than 2 cm from each other.

9. The wireless EEG recording device of claim 1, wherein the RF transmitter transmits the packet to the external receiver using an industrial, scientific, and medical (ISM) band.

10. The wireless EEG recording device of claim 9, wherein the transmitter operates in a range of 902 MHz to 928 MHz.

11. The wireless EEG recording device of claim 9, wherein the transmitter supports twelve wireless EEG recording devices within the ISM band.

12. The wireless EEG recording device of claim 1, wherein the RF transmitter transmits the packet to the external receiver using less than or equal to 200 Kbps.

13. The wireless EEG recording device of claim 1, wherein the wireless EEG recording device is located a distance less than or equal to 10 m from the external receiver.
